(19) United States
(12) Patent Application Publication
(10) Pub. No.: US 2024/0341601 A1
(43) Pub. Date: Oct. 17, 2024

(54) SURGICAL POSITIONING METHODS AND METHODS FOR DETERMINING REGIONS SUBJECT TO RADIATION

(71) Applicant: Medtronic Navigation, Inc., Louisville, CO (US)

(72) Inventors: Dany Junio, Brookline, MA (US); Rikuo Sato, Somerville, MA (US); Shreya Sridhar, Windham, NH (US); Rasika A. Parkar, Wayland, MA (US); Patrick A. Helm, Canton, MA (US)

(21) Appl. No.: 18/630,872

(22) Filed: Apr. 9, 2024

Related U.S. Application Data
(60) Provisional application No. 63/459,187, filed on Apr. 13, 2023.

(51) Int. Cl. A61B 5/00; A61B 34/10; A61B 34/20

(52) U.S. Cl. CPC A61B 5/0037; A61B 34/10; A61B 34/20; A61B 2034/104; A61B 2034/107; A61B 2034/2065

(57) ABSTRACT

A method according to at least one embodiment of the present disclosure includes: receiving, from an imaging device in a pose relative to a patient, a first two-dimensional image depicting a first region of the patient from a perspective of the pose; overlaying, over the first two-dimensional image, a virtual collimator to produce a second two-dimensional image, the second two-dimensional image depicting the first region as seen by a radiation source from the perspective of the pose; and segmenting the second two-dimensional image into at least two segments, where a first segment of the at least two segments is subject to radiation produced by the radiation source while the radiation source is in the pose, and where a second segment of the at least two segments is subject to less radiation than the first segment of the at least two segments while the radiation source is in the pose.

### CROSS-REFERENCE TO RELATED APPLICATION

This application claims the benefit of and priority to U.S. Provisional Patent Application No. 63/459,187 filed Apr. 13, 2023, the entire disclosure of which is incorporated by reference herein.

### BACKGROUND

The present disclosure is generally directed to imaging, and relates more particularly to surgical imaging.

Images may be used during a surgical operation for a variety of surgical tasks. The images may be obtained prior to and/or during a surgical operation. The imaging may be used by a medical provider for diagnostic and/or therapeutic purposes.

### BRIEF SUMMARY

Example aspects of the present disclosure include:

A method according to at least one embodiment of the present disclosure comprises: receiving, from an imaging device in a pose relative to a patient, a first two-dimensional image depicting a first region of the patient from a perspective of the pose; overlaying, over the first two-dimensional image, a virtual collimator to produce a second two-dimensional image, the second two-dimensional image depicting the first region as seen by a radiation source from the perspective of the pose; and segmenting the second two-dimensional image into at least two segments, wherein a first segment of the at least two segments is subject to radiation produced by the radiation source while the radiation source is in the pose, and wherein a second segment of the at least two segments is subject to less radiation than the first segment of the at least two segments while the radiation source is in the pose.

Any of the aspects herein, further comprising: causing the radiation source to move into the pose from a different position that is unaligned with the pose; and causing the radiation source to emit the radiation after the radiation source has been moved into the pose.

Any of the aspects herein, further comprising: rendering, to a display, a virtual representation of at least one of the first segment and the second segment.

Any of the aspects herein, wherein the virtual representation of the at least one of the first segment and the second segment comprises outlines of the at least one of the first segment and the second segment rendered over the second two-dimensional image.

Any of the aspects herein, further comprising: transforming, based on the pose of the imaging device and a second pose of the radiation source, at least one of the first segment into a third segment that is subject to radiation produced by the radiation source while the radiation source is in the second pose and the second segment into a fourth segment that is subject to less radiation than the third segment while the radiation source is in the second pose.

Any of the aspects herein, further comprising: causing the radiation source to move into the second pose; and causing the radiation source to emit radiation after the radiation source has been moved into the second pose.

Any of the aspects herein, wherein the transforming comprises: registering at least one of coordinates associated with a boundary of the first segment and coordinates associated with a boundary of the second segment into a coordinate system associated with the radiation source.

Any of the aspects herein, wherein the radiation source comprises an adjustable collimator.

Any of the aspects herein, further comprising: adjusting, based on an orientation of the adjustable collimator, a shape of the virtual collimator.

Any of the aspects herein, further comprising: rendering, to a display, a virtual representation of the virtual collimator.

A system according to at least one embodiment of the present disclosure comprises: a processor; and a memory storing data thereon that, when processed by the processor, cause the processor to: receive, from an imaging device in a first pose, an image depicting a frame mechanically coupled with a head of a patient; determine, based on the image, a pose of the head relative to the imaging device; and cause, based on the pose of the head, the imaging device to move from the first pose into a second pose to align the imaging device with the head.

Any of the aspects herein, wherein the frame comprises two or more navigation markers disposed on the frame.

Any of the aspects herein, wherein the data further cause the processor to: determine, based on the two or more navigation markers, a pose of the frame.

Any of the aspects herein, wherein the two or more navigation markers comprise optical navigation markers capable of being identified in the image.

Any of the aspects herein, wherein the data further cause the processor to: register one or more coordinates associated with the frame to a coordinate system associated with the imaging device.

Any of the aspects herein, wherein the data further cause the processor to: capture, using the imaging device, a no-fly-zone scan of the patient.

Any of the aspects herein, wherein the no-fly-zone scan comprises a first zone through which the imaging device is moveable and a second zone through which the imaging device avoids moving.

Any of the aspects herein, wherein causing the imaging device to move from the first pose to the second pose comprises:

determining a navigation path that does not pass through the second zone.

Any of the aspects herein, wherein the second zone comprises at least one of the head of the patient and the frame.

Any of the aspects herein, wherein the imaging device comprises an O-arm.

A system according to at least one embodiment of the present disclosure comprises: a processor; and a memory storing data thereon that, when processed by the processor, cause the processor to: receive, from an imaging device in a first pose relative to a patient, a first two-dimensional image depicting a first region of the patient from a perspective of the first pose; overlay, over the first two-dimensional image, a virtual collimator to produce a second two-dimensional image, the second two-dimensional image depicting a first region of the patient as seen by a radiation source from the perspective of the first pose; segment, the second two-dimensional image into at least two segments, wherein a first segment of the at least two segments is subject to radiation produced by the radiation source while the radiation source is in the first pose, and wherein a second segment of the at least two segments is subject to less radiation than the first segment of the at least two segments while the radiation source is in the first pose; determine, based on the first two-dimensional image, a pose of a head of the patient relative to the imaging device; and cause, based on the pose of the head, the imaging device to move from the first pose to a second pose to align the imaging device with the head.

A system according to at least one embodiment of the present disclosure comprises: a first imaging device; a second imaging device; a processor; and a memory coupled to the processor and storing data thereon that, when processed by the processor, enable the processor to: receive, from the first imaging device in a pose relative to a patient, a first two-dimensional image depicting a first region of the patient from a perspective of the pose; overlay, over the first two-dimensional image, a virtual collimator to produce a second two-dimensional image, the second two-dimensional image depicting the first region as seen by a radiation source from the perspective of the pose; and segment the second two-dimensional image into at least two segments, wherein a first segment of the at least two segments is subject to radiation produced by the radiation source while the radiation source is in the pose, and wherein a second segment of the at least two segments is subject to less radiation than the first segment of the at least two segments while the radiation source is in the pose.

Any of the aspects herein, wherein the data further enable the processor to: capture, using the first imaging device and the second imaging device, a three-dimensional image depicting the patient; and segment the three-dimensional image into a plurality of segments, wherein a first segment of the plurality of segments contains the first region of the patient.

Any of the aspects herein, wherein the data further enable the processor to: determine, based on the first segment of the plurality of segments, a location of the first region of the patient relative to the first imaging device; and cause, based on the location of the first region of the patient, the first imaging device to move into the pose relative to the patient.

Any of the aspects herein, wherein the data further enable the processor to: cause the radiation source to move into the pose from a different position that is unaligned with the pose; and cause the radiation source to emit the radiation after the radiation source has been moved into the pose.

Any of the aspects herein, wherein the data further enable the processor to: render, to a display, a virtual representation of at least one of the first segment and the second segment of the at least two segments.

Any of the aspects herein, wherein the virtual representation of the at least one of the first segment and the second segment of the at least two segments comprises outlines of the at least one of the first segment and the second segment of the at least two segments rendered over the second two-dimensional image.

Any of the aspects herein, wherein the data further enable the processor to: transform, based on the pose of the first imaging device and a second pose of the radiation source, at least one of the first segment of the at least two segments into a third segment that is subject to radiation produced by the radiation source while the radiation source is in the second pose and the second segment of the at least two segments into a fourth segment that is subject to less radiation than the third segment while the radiation source is in the second pose.

Any of the aspects herein, further comprising: causing the radiation source to move into the second pose; and causing the radiation source to emit radiation after the radiation source has been moved into the second pose.

Any of the aspects herein, wherein the transforming comprises: registering at least one of coordinates associated with a boundary of the first segment of the at least two segments and coordinates associated with a boundary of the second segment of the at least two segments into a coordinate system associated with the radiation source.

Any of the aspects herein, wherein the radiation source comprises an adjustable collimator.

Any of the aspects herein, wherein the data further enable the processor to: adjust, based on an orientation of the adjustable collimator, a shape of the virtual collimator.

Any of the aspects herein, wherein the data further enable the processor to: render, to a display, a virtual representation of the virtual collimator.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 shows a block diagram of components of a system according to at least one embodiment of the present disclosure;

FIG. 2 shows additional components of the system according to at least one embodiment of the present disclosure;

FIG. 3A shows aspects of imaging a patient according to at least one embodiment of the present disclosure;

FIG. 3B shows additional aspects of imaging the patient according to at least one embodiment of the present disclosure;

FIG. 3C shows aspects of imaging a patient with a virtual collimator according to at least one embodiment of the present disclosure;

FIG. 3D shows additional aspects of imaging the patient with the virtual collimator according to at least one embodiment of the present disclosure;

FIG. 3E shows segmenting an image of the patient according to at least one embodiment of the present disclosure;

FIG. 4A shows a patient on a table proximate an imaging device according to at least one embodiment of the present disclosure;

FIG. 4B shows movement of the imaging device relative to the patient according to at least one embodiment of the present disclosure;

FIG. 5 shows a flowchart according to at least one embodiment of the present disclosure;

FIG. 6 shows a flowchart according to at least one embodiment of the present disclosure;

FIG. 7 shows a flowchart according to at least one embodiment of the present disclosure; and

FIG. 8 shows a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Imaging devices, such as an O-arm, C-arm, x-ray source and receiver, and the like may be used to capture one or more images of a patient before and/or during a surgery or surgical procedure. In some cases, the images may depict the head of the patient, and may include three-dimensional (3D) scans of the patient's head (or portions thereof). In these cases, knowing where the patient's head is in relation to the O-arm or other imaging device may reduce the set-up time needed to perform the scan and/or reduce the amount of risk or discomfort associated with the patient.

Aspects of the present disclosure provide options for aligning a patient's head to an O-arm canter or, more generally, to a portion of an imaging device. In some embodiments, image processing may be utilized on images generated by red-green-blue (RGB) cameras or red-green-blue-depth (RGBD) cameras, which are RGB cameras that also include depth data. The image processing may localize the patient's head or a frame connected to the patient's head. Once the head or frame is localized, the O-arm or other imaging device may be moved to the desired position, such that the O-arm or other imaging device aligns with the patient's head.

In some embodiments, a navigation attachment may be attached to a frame connected to the patient's head. Optical navigation may then be used to identify the navigation attachment and register coordinates associated with the navigation attachment to O-arm space, allowing the O-arm to be navigated to align with the patient's head.

In some embodiments, the O-arm or other imaging device may perform a no-fly-zone scan of the patient. The no-fly-zone may allow a navigation system to determine the location of the patient's head, as well as any other obstacles (e.g., frames connected to the patient's head) the O-arm should avoid contact with while moving. The O-arm may then be moved to align with the patient's head.

When capturing two-dimensional (2D) images using the O-arm or other imaging device, an indication of which portions of the patient are subject to radiation may be needed prior to actual x-ray exposure.

Aspects of the present disclosure enable determining which portions of a patient are subject to radiation prior to x-ray exposure. By positioning a camera in the same position as an x-ray source would be, and applying a virtual overlay (e.g., a virtual collimator) to the camera, the resulting image depicts the patient anatomy subject to radiation. In some embodiments, one or more algorithms may be used to modify the virtual overlay, such as when the camera is mounted somewhere other than the source's location. In some embodiments, the overlay may be updated or changed based on a patient's distance from the camera.

In some embodiments, a camera may capture a 2D image, and an overlay may be applied to the 2D image, such that the area within the overlay represents a region of a patient that would be subject to an x-ray beam emitted from a radiation source. The overlay may represent regions that would be subject to less radiation due to a collimator disposed on the radiation source.

Embodiments of the present disclosure beneficially reduce the amount of collimator adjustment necessary to capture images of patients. The use of the virtual collimator also permits later improvements through software.

A pixel on the virtual overlay represents a single straight line of site, and all lines of sight represented by the pixel must converge on the camera. As a result, the lines of sight are neither parallel nor skew, so any mark on the pixel will mark a single line of sight that is entering the focal point at an angle, the boundaries of the cross sections of the image define the bounds of the x-ray beam, with the boundaries lining up on a straight line back to the camera. In the image, the lines are represented as dots (e.g., pixels), so a dot in the image represents a line in the real world, as well as the boundaries of all cross sections of the x-ray beam. Therefore, a line in the image represents a plane in the real world at an angle which could match up with a side of the boundary of each cross section (e.g., the side of the x-ray beam).

In some embodiments, a software overlay can be used to determine what portions of a patient will be subject to radiation by a radiation source during a 2D x-ray without needing depth information. By using a direct line of sight approach, whatever regions of the patient that can be seen out of the opening of the collimator from the x-ray source's perspective will be subject to radiation. As discussed herein, a camera can be provided with an overlay that replicates the filter openings of a collimator as if the camera were in the source's position. What the camera views through the overlay will be what the x-ray source sees through the filter. After positioning using the overlay, the source can be moved (e.g., using a gantry) into the same position as the camera. In some embodiments, the difference in camera and x-ray source positions can be compensated for with the movement of the source and software used by a navigation system. The distance between the x-ray source and the filter can be known, and the edges of the x-ray beam exiting through the filter may be roughly tracked back to a single focal point.

Embodiments of the present disclosure provide technical solutions to one or more of the problems of (1) determining which portions of a patient will be subject to radiation prior to actual x-ray exposure and (2) aligning an O-arm or other imaging device with a patient.

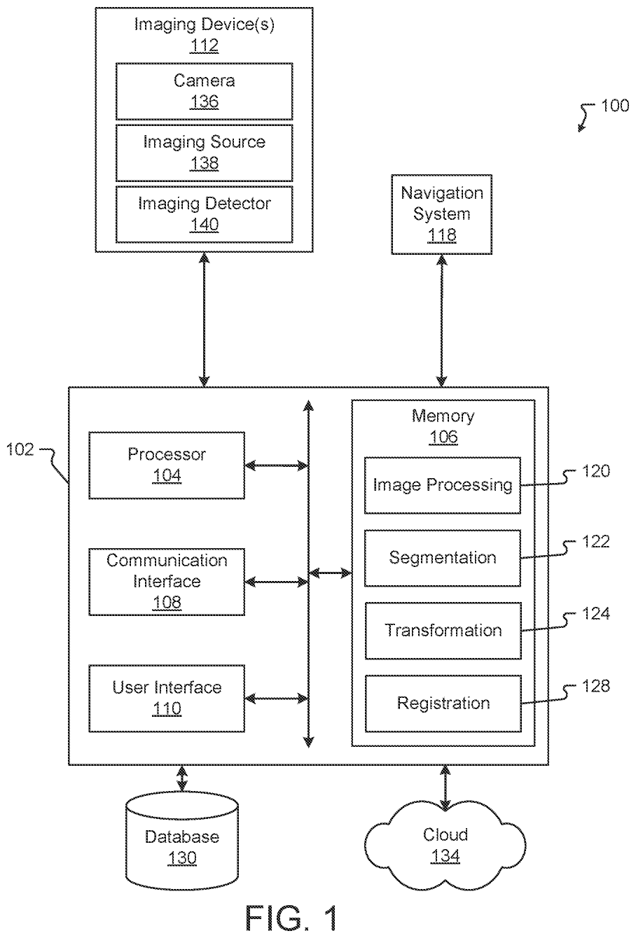

Turning first to FIG. 1, a block diagram of a system 100 according to at least one embodiment of the present disclosure is shown. The system 100 may be used to align an O-arm or other imaging device with a patient (or portions of the patient); to determine which portions of a patient will be subject to radiation prior to actual x-ray exposure; to control, pose, and/or otherwise manipulate a surgical mount system, a surgical arm, and/or other surgical tools attached thereto; and/or to carry out one or more other aspects of one or more of the methods disclosed herein. The system 100 comprises a computing device 102, one or more imaging devices 112, a navigation system 118, a database 130, and/or a cloud or other network 134. Systems according to other embodiments of the present disclosure may comprise more or fewer components than the system 100. For example, the system 100 may not include one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 500, 600, 700, and/or 800 described herein, or of any other methods. The memory 106 may store, for example, instructions and/or machine learning models that support one or more functions of one or more components of the system 100. For instance, the memory 106 may store content (e.g., instructions and/or machine learning models) that, when executed by the processor 104, enable image processing 120, segmentation 122, transformation 124, and/or registration 128. Such content, if provided as in instruction, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the database 130, and/or the cloud 134.

The computing device 102 may also comprise a communication interface 108. The communication interface 108 may be used for receiving image data or other information from an external source (such as the imaging device 112, the navigation system 118, the database 130, the cloud 134, and/or any other system or component not part of the system 100), and/or for transmitting instructions, images, or other information to an external system or device (e.g., another computing device 102, the imaging device 112, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computing device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

In some embodiments, the imaging device 112 comprises a camera 136, an imaging source 138, and an imaging detector 140. The camera 136 may comprise one or more imaging features discussed herein with respect to the imaging device 112. In other words, the camera 136 may operate to capture image data, which may be used by the processor 104 (e.g., using image processing 120) to generate an image from the image data. As discussed further below, the camera 136 may be used in determining which segments of the generated image depict patient anatomy subject to various radiation levels, such as radiation generated by the imaging source 138 and the imaging detector 140 when capturing an image of the patient or patient anatomy.

The imaging source 138 generates or otherwise emits radiation, waves, or other signals that are received or captured by the imaging detector 140 to generate an image of the anatomical elements (e.g., patient anatomy) positioned therebetween.

The imaging source 138 and/or the imaging detector 140 may each comprise a collimator 144. The collimator 144 aligns the X-rays or other signals passing therethrough (e.g., X-rays generated by the imaging source, X-rays captured by the imaging detector, and so forth) to, for example, improve the resulting image. In some embodiments, the collimator 144 may comprise an open portion and a closed portion. The open portion may be or comprise a portion of the collimator 144 through which X-rays or other signals may pass, and through which the passing X-rays are focused or aligned. The closed portion may be or comprise a portion of the collimator 144 through which X-rays or other signals are blocked or prevented from passing.

In some embodiments, the collimator 144 may comprise one, two, three, or more degrees of freedom. For instance, the collimator 144 may comprise three degrees of freedom, with the collimator 144 capable of opening or closing one or more shutters in a first direction (e.g., an X-axis direction) and a second direction (e.g., a Y-axis direction), while also capable of rotating the shutters independently of one another such that an open portion of the collimator 144 (e.g., the portion through which the X-rays are focused) is capable of rotating in a first plane (e.g., in an XY-plane). In some embodiments, the shutters may be controlled by the one or more motors disposed in the imaging source 138 or the imaging detector 140.

The navigation system 118 may provide navigation for a surgeon during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors. In various embodiments, the navigation system 118 may be used to track a position and orientation (e.g., a pose) of the imaging device 112, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. In some embodiments, the system 100 can operate without the use of the navigation system 118. The navigation system 118 may be configured to provide guidance to a surgeon or other user of the system 100 or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 500, 600, 700, and/or 800 described herein. The system 100 or similar systems may also be used for other purposes.

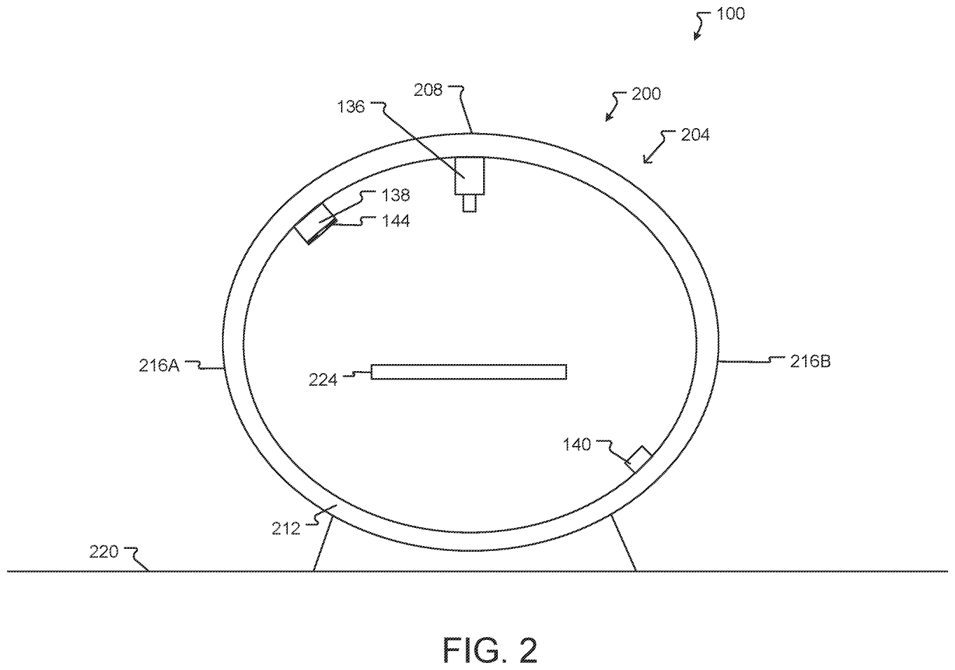

FIG. 2 shows a robotic platform 200 according to at least one embodiment of the present disclosure. In some embodiments, the robotic platform 200 may be part of the system 100. The robotic platform 200 includes a support structure 204 and an operating table 224. The support structure 204 comprises an upper wall or member 208, a lower wall or member 212, and a pair of sidewalls or members 216A, 216B. In some embodiments, the table 224 is positioned orthogonally to the support structure 204, such that the table 224 may extend in a first direction from the support structure 204. In some embodiments, the table 224 may be mounted to the support structure 204. In other embodiments, the table 224 may not be mounted to the support structure 204 and may be mounted elsewhere.

In some embodiments, the support structure 204 is fixed securable to an operating room wall 220 (such as, for example, a ground surface of an operating room or other room). In other embodiments, the support structure 204 may be releasably securable to the operating room wall 220 or may be a standalone component that is simply supported by the operating room wall 220. In some embodiments, the table 224 may be mounted to the support structure 204. In other embodiments, the table 224 may be releasably mounted to the support structure 204. In still other embodiments, the table 224 may not be attached to the support structure 204. In such embodiments, the table 224 may be supported and/or mounted to an operating room wall, for example. In embodiments where the table 224 is mounted to the support structure 204 (whether detachably mounted or permanently mounted), the table 224 may be mounted to the structure 204 such that a pose of the table 224 relative to the structure 204 is selectively adjustable.

The table 224 may be any operating table 224 configured to support a patient during a surgical procedure. The table 224 may include any accessories mounted to or otherwise coupled to the table 224 such as, for example, a bed rail, a bed rail adaptor, an arm rest, an extender, or the like. The operating table 224 may be stationary or may be operable to maneuver a patient (e.g., the operating table 224 may be able to move). In some embodiments, the table 224 has two positioning degrees of freedom and one rotational degree of freedom, which allows positioning of the specific anatomy of the patient anywhere in space (within a volume defined by the limits of movement of the table 224). For example, the table 224 can slide forward and backward and from side to side, and can tilt (e.g., around an axis positioned between the head and foot of the table 224 and extending from one side of the table 224 to the other) and/or roll (e.g., around an axis positioned between the two sides of the table 224 and extending from the head of the table 224 to the foot thereof). In other embodiments, the table 224 can bend at one or more areas (which bending may be possible due to, for example, the use of a flexible surface for the table 224, or by physically separating one portion of the table 224 from another portion of the table 224 and moving the two portions independently). In at least some embodiments, the table 224 may be manually moved or manipulated by, for example, a surgeon or other user, or the table 224 may comprise one or more motors, actuators, and/or other mechanisms configured to enable movement and/or manipulation of the table 224 by a processor such as the processor 104.

The robotic platform 200 also comprises the imaging source 138 and the imaging detector 140. In some embodiments, both the imaging source 138 and the imaging detector 140 may be moveable via the robotic platform 200. For example, the support structure 204 may be rotatable, and may rotate to change the position of the imaging source 138 and the imaging detector 140. In some embodiments, the imaging source 138 and the imaging detector 140 may be moveable by a gantry, such that the imaging source 138 and the imaging detector 140 can be rotated 360 degrees around the table 224 (and around any patient on the table 224). Additionally or alternatively, the robotic platform 200 and/or components thereof, such as the gantry that positions the imaging source 138 and the imaging detector 140, can be moved laterally along the table 224 (e.g., in a direction that points out of the page of FIG. 2), such that a full body scan of the patient can be captured.

In some embodiments, the camera 136 may be disposed in a known position relative to the imaging source 138 and/or the imaging detector 140. In such embodiments, coordinates associated with the camera 136, the imaging source 138, and/or the imaging detector 140 may be registered into a common coordinate system. Additionally or alternatively, coordinates associated with the camera 136 may be registered into a coordinate system associated with the imaging source 138 and/or the imaging detector 140, or vice versa. Based on the known distances between the camera 136, the imaging source 138, and the imaging detector 140, the navigation system 118 may be able to maneuver the camera 136, the imaging source 138, and the imaging detector 140 relative to one another while avoiding collisions therebetween. In some embodiments, the camera 136 may capture a first image in a first pose, while the imaging detector 140 is disposed in a second pose different from the first pose. In such cases, the camera 136 may be moved out of the first pose after capturing the first image, and the imaging detector 140 may be moved into the first pose. Alternatively, the camera 136 may remain in a fixed location throughout one or more portions of the surgery or surgical procedure, such that the imaging detector 140 cannot move into the first pose. In such cases, the imaging detector 140 may begin in a predetermined pose relative to the camera 136, and may be moved relative to the camera 136, with the navigation system 118 tracking the position of the imaging detector 140 relative to a known coordinate system and/or relative to the camera 136, such that the navigation system 118 knows that position of the camera 136, the position of the imaging detector 140, and the position of the imaging detector 140 relative to the camera 136.

In some embodiments, the platform 200 may be used to localize patient anatomy for a surgery or surgical procedure. The platform 200 may perform a three-dimensional scan of a patient located in the table 224. For example, the navigation system 118 may cause the imaging source 138 and the imaging detector 140 to perform a 360 degree image capture of the patient on the table 224. Additionally or alternatively, the platform 200 or components thereof may be able to move along a length of the patient (e.g., along a length of the table on which the patient rests) to capture one or more images of the patient. In some embodiments, the platform 200 may move the imaging source 138 and the imaging detector 140 in a 360 degree rotation around the patient as well as along the lateral direction of the patient to generate a three-dimensional image of the patient. The three-dimensional image of the patient may be captured by one or more imaging devices, such as by the imaging source 138 and the imaging detector 140, the camera 136, other imaging devices not associated with the platform 200, combinations thereof, and the like. The computing device 102 may use the captured images to localize the patient anatomy that is subject to the surgery or surgical procedure. For example, the computing device 102 may use image processing 120 and segmentation 122 to process and then segment the captured images into one or more segments. The computing device 102 may then identify, based on the segmenting, the target patient anatomy for the surgical procedure. In some embodiments, the computing device 102 may render the segmented images to a display (e.g., the user interface 110), and the user (e.g., a surgeon) may select the target patient anatomy. After the target patient anatomy has been designated, one or more components of the platform 200 (e.g., the imaging source 138 and the imaging detector 140) may be moved to align relative to the target patient anatomy. For example, the platform 200 may move laterally relative to the patient based on the target patient anatomy, such that the camera 136 is positioned above and can capture images depicting the region of the patient with the target patient anatomy.

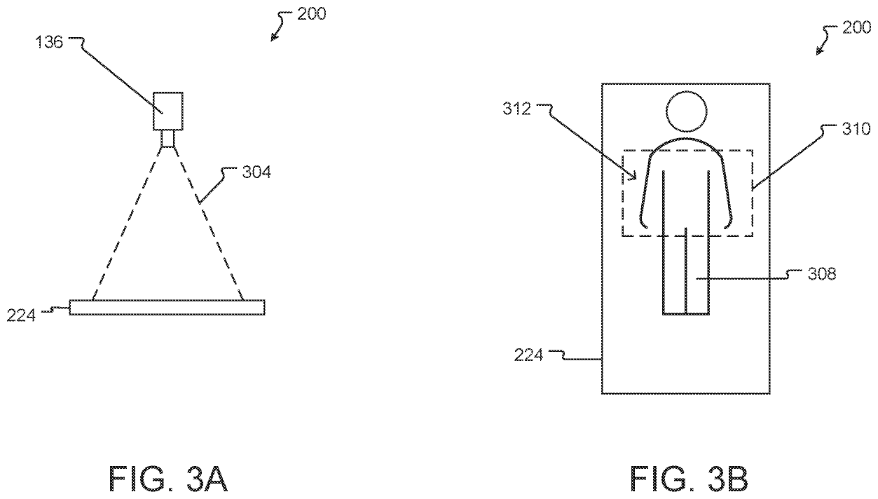

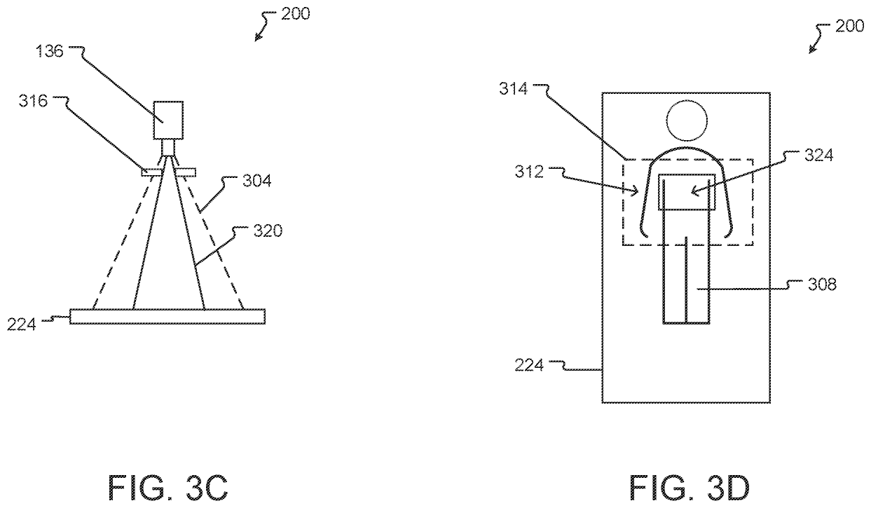

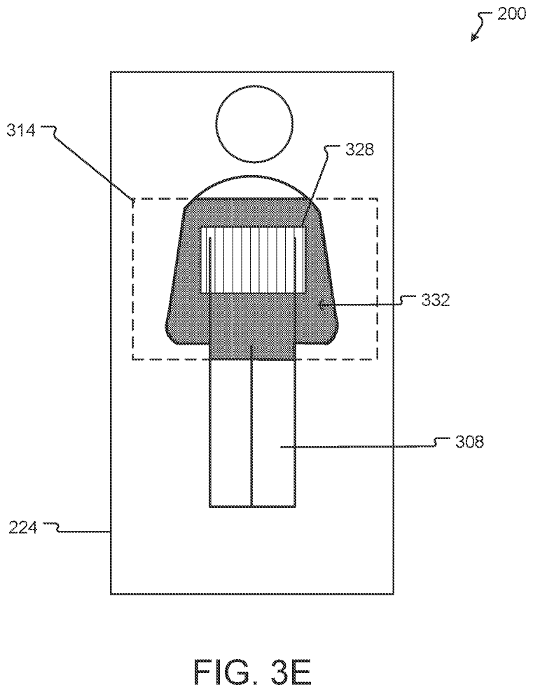

FIGS. 3A-3E illustrate aspects of imaging a patient according to at least one embodiment of the present disclosure. The aspects depicted may relate to imaging using, for example, the imaging device 112 (e.g., an O-arm, an imaging source and imaging detector, a camera, etc.). In some embodiments, the patient may be imaged using the robotic platform 200 (e.g., using the camera 136, using the imaging source 138 and the imaging detector 140, etc.).

FIG. 3A shows a field of view 304 of the camera 136. The field of view 304 may represent the area or volume of space that can be seen and captured by the camera 136. As shown in FIG. 3B, the camera 136 may capture a first image 310 of a patient 308 based on the field of view 304. In some embodiments, the first image 310 may be or comprise a 2D image of the patient. The first image 310 includes a first region 312 of the patient 308 that can be seen by the camera 136 based on the field of view 304. In other words, the field of view 304 limits the camera 136 to capturing image data associated with the first region 312 of the patient 308, while the remainder of the patient 308 is not depicted in the first image 310. In some embodiments, the field of view 304 may be adjustable. In these embodiments, the field of view 304 may be capable of being adjusted (e.g., increased or enlarged, decreased or shrunk, moved, etc.) such that the first region 312 changes in size, and the resulting first image 310 depicts a different region of the patient 308.

In some cases, the field of view 304 of the camera 136 may be adjusted, changed, or otherwise manipulated, such as when the camera 136 is being used to determine which regions of the patient 308 are or will likely be subject to radiation. As shown in FIGS. 3C-3D, a virtual collimator 316, when overlaid on the first image 310, may effectively operate to change the field of view 304 of the camera 136. The virtual collimator 316 may block certain portions of the first image 310, such that the camera 136 appears to have a collimated field of view 320. In other words, the overlay of the virtual collimator 316 on the first image 310 results in an image similar to what would be captured by the camera 136 if a real collimator had been disposed over the camera 136. In some embodiments, the collimated field of view 320 may be a narrower field of view than the field of view 304, such that the resulting image captured by the camera 136 may depict fewer and/or smaller regions of the patient 308 than the image created when the camera 136 has the field of view 304.

The adjustment of the field of view 304 using the virtual collimator 316 may be done virtually (e.g., by the processor 104 using segmentation 122). As an example, the camera 136 may capture image data that is processed to create the first image 310. Then, the first image 310 may be adjusted by overlaying the virtual collimator 316 onto the first image 310. The overlaying of the virtual collimator 316 may result in a second image 314. The second image 314 may also be a 2D image, and may depict information contained in the first image 310, as well as additional information related to how the virtual collimator 316 affects the field of view of the camera 136. As depicted in FIG. 3D, the overlaying of the virtual collimator 316 results in the second image 314, which includes the first region 312 and a second region 324. While the first region 312 depicts what is captured by the camera 136 with the field of view 304, the second region 324 depicts what would be captured by the camera 136 if the collimator 144 were positioned between the camera 136 and the patient 308 at the pose. The second region 324 depicts the region of the first image 310 that would be subject to radiation if the camera 136 were instead the imaging source 138 with the collimator 144 positioned over the imaging source 138. In other words, by overlaying the virtual collimator 316 on the first image 310 to produce the second image 314, the portions of the patient subject to various levels of radiation can be predicted.

The second image 314 may be segmented into one or more segments or sections. The different segments may represent areas that would be subject to different amounts of radiation if the imaging source 138 were aligned in the same pose as the camera 136. As depicted in FIG. 3E, the second image 314 may be segmented into two or more segments.

The two or more segments may include a first segment 328 and a second segment 332. The first segment 328 may be or comprise the first region 312 of the patient 308. The first segment 328 may depict or represent the region of the patient that is subject to or is likely to experience reduced radiation when the imaging source 138 emits radiation while aligned where the camera 136 was to capture the first image 310 and/or the second image 314. Stated differently, the first segment 328 may represent portions of the patient 308 that were within the field of view 304, but were hidden from view in the collimated field of view 320 based on the alignment of the virtual collimator 316 with the camera 136. The second segment 332 may depict or represent the region of the patient 308 that is less likely to experience radiation or is likely to experience a reduced amount of radiation when the imaging source 138 emits radiation while aligned where the camera 136 was to capture the first image 310 and/or the second image 314. In other words, the second segment 332 may represent portions of the patient 308 that were in both the field of view 304 and the collimated field of view 320.

The segmenting may be performed by one or more components of the system 100. For example, the processor 104 may use segmentation 122 to segment the second image 314. In some embodiments, the segmentation 122 may be or comprise an algorithm designed to segment the second image 314. Examples of algorithms that may be used include, but are in no way limited to, edge detection algorithms, classification algorithms, threshold segmentation algorithms, clustering algorithms (e.g., k-means clustering), combinations thereof, and the like. Additionally or alternatively, the segmentation 122 may include one or more machine learning and/or artificial intelligence data models trained on historical data to segment the second image 314 into one or more segments. For example, the segmentation 122 may be or comprise a neural network (e.g., a Convolutional Neural Network (CNN), a Recurrent Neural Network (RNN), etc.) trained on data comprising similar segmented images, such that the segmentation 122 can receive image data associated with the second image 314 (including the second image 314 itself) and produce a segmented version of the second image 314.

In some embodiments, information associated with any one or more of the first image 310, the second image 314, the virtual collimator 316, the first segment 328, the second segment 332 may be rendered to a display, such as to the user interface 110. For example, the second image 314, the first segment 328, and the second segment 332 may be rendered as virtual representations to the user interface 110 so that a physician or member of the surgical staff can view which segments will be subject to different amounts of radiation. The virtual representations may include visual indicators that separate and/or distinguish the first segment 328 and the second segment 332 from one another (and, more generally, separate other segments of the second image 314 from one another). For example, the first segment 328 and the second segment 332 may each include an outline around the border thereof (e.g., a red outline) to help a user visually distinguish which portions of the patient 308 belong to which segment. Additionally or alternatively, the virtual collimator 316 may be visually rendered over the first image 310, enabling the user to more easily determine the location of the virtual collimator 316 relative to the camera 136, the patient 308, and/or the like.

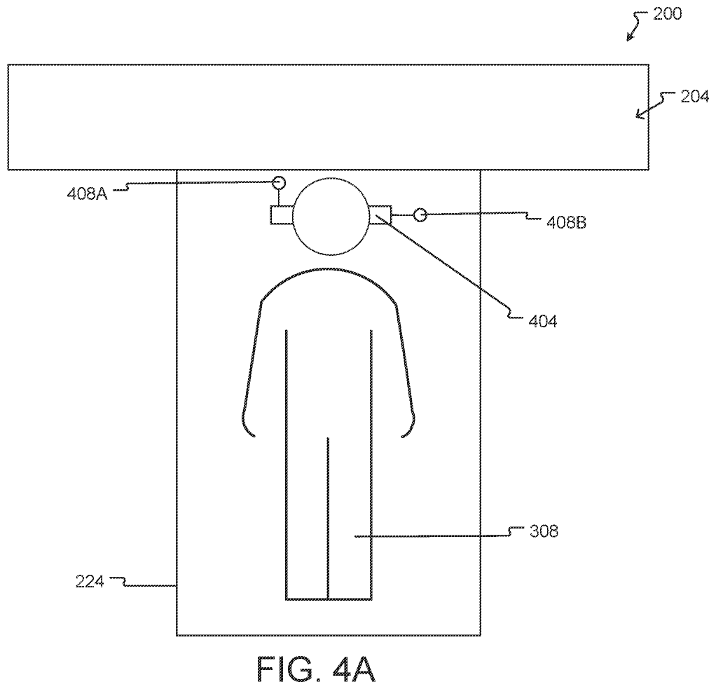

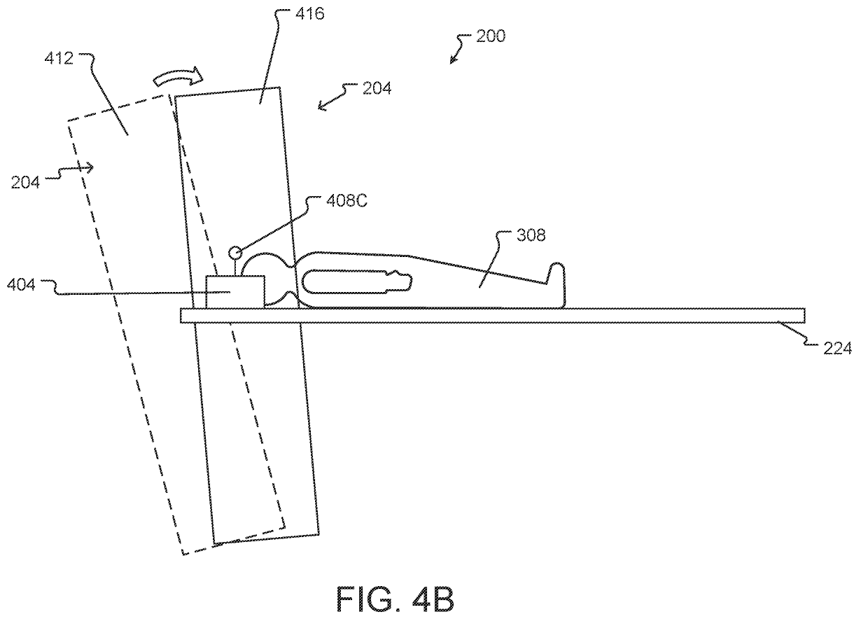

FIGS. 4A-4B illustrate the patient 308 being imaged by and aligned with portions of the platform 200 according to at least one embodiment of the present disclosure. The platform 200 may be able to adjust the imaging device 112, the support structure 204, and/or the table 224 to align the imaging device 112 with the patient 308.

The platform 200 includes a stereotactic frame 404 mounted to the table 224 and/or the patient 308. The stereotactic frame 404 may facilitate navigation of the imaging device 112 relative to patient 308 by enabling the navigation system 118 to identify the stereotactic frame 404 and/or the patient 308 in a known coordinate system. In some embodiments, the stereotactic frame 404 may be mechanically coupled to the patient 308, such as to the patient's head. The stereotactic frame 404 includes a plurality of navigation markers 408A-408C that can be used by the navigation system 118 to identify a pose of the stereotactic frame 404 relative to the patient 308. While there are three navigation markers 408A-408C depicted in FIGS. 4A-4B, it is to be understood that an additional or alternative number of navigation markers is possible. Based on the pose of the stereotactic frame 404 and a known pose of the stereotactic frame 404 relative to the patient 308, the computing device 102 and/or the navigation system 118 can determine the pose of portions of the patient 308 (e.g., the patient's head) relative to the imaging device 112.

The imaging device 112 may capture an initial image of the patient 308 positioned on the table 224 with the stereotactic frame 404 coupled with the patient's head. The image may be captured by the imaging device(s) 112 connected to the support structure 204 of the platform 200 (e.g., the imaging source 138 and imaging detector 140, the camera 136, etc.). In some embodiments, the initial image may be formed by the processor 104 using image processing 120 to generate an image from the image data captured by the imaging device 112. The image may depict the patient 308, the stereotactic frame 404, and the navigation markers 408A-408C. The navigation markers 408A-408C may be radiopaque or optical markers, such that the navigation markers 408A-408C are identifiable in the initial image. The processor 104 may use segmentation 122 to identify each navigation marker of the three navigation markers 408A-408C, and use the identified navigation markers 408A-408C to determine the pose of the imaging device 112 relative to the patient 308. For example, the image processing 120 may be or comprise an algorithm that identifies radiopaque elements in image data (e.g., elements with a pixel value above or below a predetermined value are labeled by the algorithm as radiopaque).

Based on the identified radiopaque elements, the processor 104 may use registration 128 to transform the coordinates associated with the navigation markers 408A-408C into a coordinate system associated with the imaging devices 112 and/or the patient 308. Additionally or alternatively, due to the predetermined pose of the navigation markers 408A-408C relative to the stereotactic frame 404, the registration 128 may register one or more coordinates of the stereotactic frame 404 into the coordinate system. As a result, the navigation system 118 knows the pose of the stereotactic frame 404, as well as the pose of the patient 308 relative to the imaging devices 112. The navigation system 118 can then navigate components of the robotic platform 200, such as the imaging devices 112, such that the components avoid contacting the stereotactic frame 404 and/or the patient 308.

In some embodiments, the platform 200 may perform a no-fly-zone scan of the patient 308. The no-fly-zone scan may include the platform 200 beginning the scan at the end of the table 224, moving along the table 224 toward the patient's head, and ending at the other end of the table 224. The scan may generate a point cloud of data from which the location of the patient's head can be identified. In some embodiments, the no-fly-zone scan may be performed in addition to the initial image capture to supplement the navigation system 118 in navigating components with respect to the patient's head, while in other embodiments the no-fly-zone scan may be performed as an alternative to the initial image capture. The processor 104 may use segmentation 122 to segment the point cloud into one or more zones. For example, the segmentation 122 may separate the scan into a first zone through which the navigation system 118 can maneuver components of the platform 200, and a second zone through which the navigation system 118 is prohibited from maneuvering components. In one embodiment, the second zone may comprise the head of the patient 308, other portions of the patient 308, the stereotactic frame 404 (and/or components thereof), combinations thereof, and the like.

Once the pose of the patient's head has been determined, the platform 200 may move such that the imaging devices 112 align with the patient's head. For example, the support structure 204 may be moved from a first orientation 412 to a second orientation 416. The second orientation 416 may be one such that the imaging device 112 aligns with the patient's head. In some embodiments, the patient's head may be positioned at the isocenter of the platform 200 when the platform 200 is in the second orientation 416.

In some embodiments, the movement of the platform 200 may include the processor 104 generating one or more navigation paths for the platform 200 in moving from the first orientation 412 to the second orientation 416. The navigation paths may be generated by the processor 104 based on information generated by the no-fly-zone scan and/or the registration of the stereotactic frame 404 and/or the patient 308 to a known coordinate system. For example, the processor 104 may determine, based on the segmentation of the no-fly-zone scan into the first zone and the second zone, that the navigation path should avoid crossing into the second zone. Additionally or alternatively, the processor 104 may determine that the navigation path should not pass within a threshold distance of the second zone.

Once the imaging device 112 has been aligned with the patient's head, one or more images of the patient's head may be captured. The use of the initial image capture and/or the no-fly-zone scan may beneficially enable the platform 200 to align with the patient's head such that the patient's head is positioned in the isocenter of the platform 200, beneficially enhancing the quality of the images of the patient's head and beneficially reducing operation costs, time, and patient exposure to radiation.

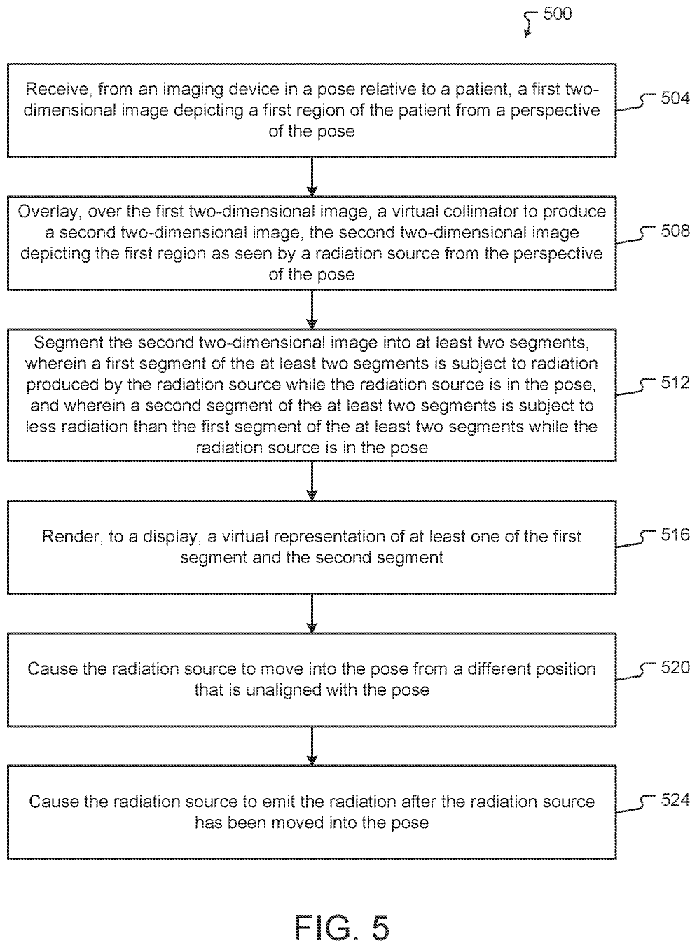

FIG. 5 depicts a method 500 that may be used, for example, to determine which regions of a patient will be subject to radiation.

The method 500 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 500. The at least one processor may perform the method 500 by executing elements stored in a memory such as the memory 106. The elements stored in memory and executed by the processor may cause the processor to execute one or more steps of a function as shown in method 500. One or more portions of a method 500 may be performed by the processor executing any of the contents of memory, such as an image processing 120, a segmentation 122, a transformation 124, and/or a registration 128.

The method 500 comprises receiving, from an imaging device in a pose relative to a patient, a first two-dimensional image depicting a first region of the patient from a perspective of the pose (step 504). In some embodiments, the first 2D image may be similar to or the same as the first image 310 discussed above. As discussed above, the first image 310 may be captured by the camera 136 based on the field of view 304, and may depict the first region 312 of the patient. In some embodiments, the first 2D image may be received after the processor 104 has used image processing 120 to generate the first 2D image from image data gathered by the camera 136.

The method 500 also comprises overlaying, over the first two-dimensional image, a virtual collimator to produce a second two-dimensional image, the second two-dimensional image depicting the first region as seen by a radiation source from the perspective of the pose (step 508). In some embodiments, the virtual collimator may be similar to or the same as the virtual collimator 316, while the second 2D image may be similar to or the same as the second image 314. As discussed above, the virtual collimator 316 may be the software or virtual representation of the collimator 144 disposed on the imaging source 138 or the imaging detector 140, such that the second image 314 depicts the perspective of which portions of the patient would be seen by and exposed to radiation if the imaging source 138 or imaging detector 140 were in the same pose as the camera 136 when the first image 310 was captured.

The method 500 also comprises segmenting the second two-dimensional image into at least two segments, wherein a first segment of the at least two segments is subject to radiation produced by the radiation source while the radiation source is in the pose, and wherein a second segment of the at least two segments is subject to less radiation than the first segment of the at least two segments while the radiation source is in the pose (step 512). The segmenting may include the processor 104 using segmentation 122 to segment the second 2D image (e.g., second image 314) into the at least two segments. In some embodiments, the at least two segments may comprise the first segment 328 and the second segment 332. As discussed above, the first segment 328 comprises the portion of the patient subject to radiation produced by the imaging source 138, while the second segment 332 comprises portions of the patient subject to less radiation than the first segment 328 at least partially due to the positioning of the collimator 144. In other words, the first segment 328 depicts an area subject to radiation emitted by the imaging source 138, while the second segment 332 represents areas that are shielded by or are otherwise subject to reduced radiation due to the collimator 144.

The method 500 also comprises rendering, to a display, a virtual representation of at least one of the first segment and the second segment (step 516). In some embodiments, the display may be part of the user interface 110. As discussed above, the virtual representation of the first segment and the second segment may include visual indicators (e.g., borders or outlines) that enable a user to identify the locations of the first segment and the second segment relative to the patient (or portions thereof such as specific anatomical structures within the patient).

The method 500 also comprises causing the radiation source to move into the pose from a different position that is unaligned with the pose (step 520). The radiation source may be similar to or the same as the imaging source 138. In the step 516, the camera 136 that was initially positioned in the pose and used to capture the first 2D image may be moved out of the pose to another location or pose (e.g., moved away from the patient, rotated to a different pose relative to the patient, detached from the support structure 204 of the platform 200, etc.) before the radiation source is moved into the pose. The radiation source may then be moved into the pose. The radiation source may be moved using one or more motors driving components of the robotic platform 200, for example.

The method 500 also comprises causing the radiation source to emit the radiation after the radiation source has been moved into the pose (step 524). The radiation source emits the radiation from the pose. The collimator 144 on the radiation source (e.g., the imaging source 138) and/or the radiation detector (e.g., imaging detector 140) may be positioned such that patient anatomy depicted in the first segment 328 of the second image 314 receive radiation from the radiation source, while patient anatomy depicted in the second segment 332 of the second image 314 experience reduced radiation dosages (e.g., due to positioning of the collimator 144).

The present disclosure encompasses embodiments of the method 500 that comprise more or fewer steps than those described above, and/or one or more steps that are different than the steps described above.

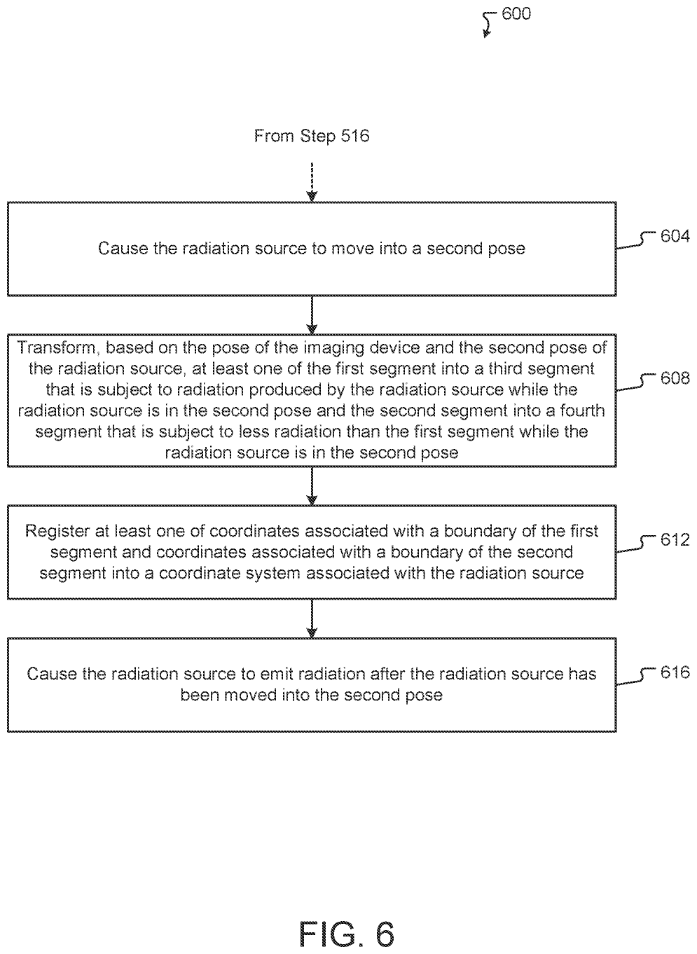

FIG. 6 depicts a method 600 that may be used, for example, to adjust image segmentation based on a pose of a radiation source.

The method 600 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 600. The at least one processor may perform the method 600 by executing elements stored in a memory such as the memory 106. The elements stored in memory and executed by the processor may cause the processor to execute one or more steps of a function as shown in method 600. One or more portions of a method 600 may be performed by the processor executing any of the contents of memory, such as an image processing 120, a segmentation 122, a transformation 124, and/or a registration 128.

The method 600 comprises causing the radiation source to move into a second pose (step 604). In some embodiments, the step 604 may continue from the step 516 of the method 500. For example, in cases where it is not desirable to move the camera 136 out of the pose and to move the radiation source into the pose (e.g., due to physical constraints such as the radiation source being too large to position in the pose in which the camera 136 was initially positioned), the radiation source may be alternatively positioned in the second pose. In some embodiments, the radiation source may begin the surgery or surgical procedure in the second pose, such as when surgical staff positions the radiation source during preoperative setup.

The method 600 also comprises transforming, based on the pose of the imaging device and the second pose of the radiation source, at least one of the first segment into a third segment that is subject to radiation produced by the radiation source while the radiation source is in the second pose and the second segment into a fourth segment that is subject to less radiation than the first segment while the radiation source is in the second pose (step 608). In some embodiments, the imaging device may be or comprise the camera 136, and the radiation source may be or comprise the imaging detector 140. Since the first segment and the second segment are based on the segmenting of the first image that was captured by the camera 136 in the pose, the first segment and the second segment depict areas of the patient subject to radiation when the radiation source is disposed in the pose. However, since the radiation source is in the second pose, the first segment and the second segment may not accurately depict the areas of the patient subject to radiation when the imaging source 138 emits radiation in the second pose. To improve the depiction of areas of the patient subject to radiation, the first and second segments may be transformed based on the known difference in pose between the camera 136 and the imaging detector 140. For example, the processor 104 may use transformation 124 to transform the first and second segments into respective third and fourth segments, which represent the first and second segments as viewed from the second pose instead of the pose.

The method 600 also comprises registering at least one of coordinates associated with a boundary of the first segment and coordinates associated with a boundary of the second segment into a coordinate system associated with the radiation source (step 612). Continuing from the step 608, the transforming of the first segment and the second segment into the third segment and the fourth segment respectively may include registering the coordinates associated with the first segment and/or the second segment into coordinates associated with the radiation source. For example, the processor 104 may use registration 128 to register coordinates associated with the first segment into a coordinate system associated with the radiation source, with such boundary coordinates representing the boundary of the third segment. Similarly, coordinates associated with the second segment may be registered using registration 128 into the coordinate system associated with the radiation source, such that the registered coordinates represent the boundary of the fourth segment. In some embodiments, the registered coordinates for both the first segment and the second segment may comprise boundary coordinates (e.g., coordinates that outline the segments and define the boundaries thereof). In some embodiments, virtual representations of the third and fourth segments may be rendered to a display, such as the user interface 110.

The method 600 also comprises cause the radiation source to emit radiation after the radiation source has been moved into the second pose (step 616). The step 616 may occur after the radiation source has been moved into the second pose, and after the first and second segments have been transformed. Due to the transformation, the third and fourth segments represent which regions of the patient will be subject to various quantities of radiation when the radiation source emits radiation while in the second pose.

The present disclosure encompasses embodiments of the method 600 that comprise more or fewer steps than those described above, and/or one or more steps that are different than the steps described above.

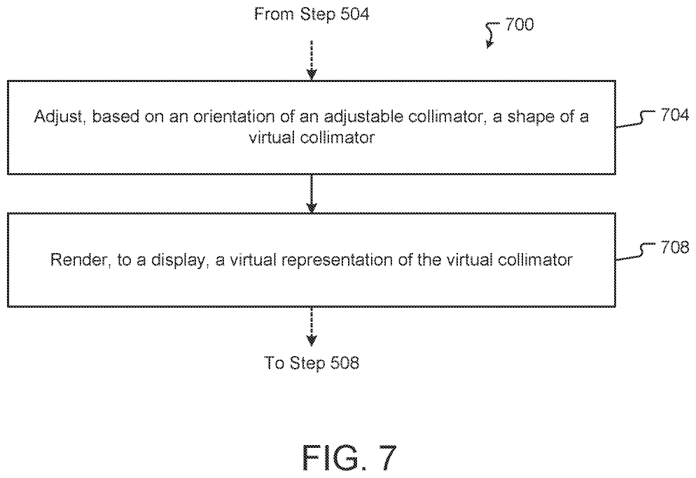

FIG. 7 depicts a method 700 that may be used, for example, to adjust a virtual collimator based on an orientation of an adjustable collimator.

The method 700 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 700. The at least one processor may perform the method 700 by executing elements stored in a memory such as the memory 106. The elements stored in memory and executed by the processor may cause the processor to execute one or more steps of a function as shown in method 700. One or more portions of a method 700 may be performed by the processor executing any of the contents of memory, such as an image processing 120, a segmentation 122, a transformation 124, and/or a registration 128.

The method 700 comprises adjusting, based on an orientation of an adjustable collimator, a shape of a virtual collimator (step 704). The step 704 may, in some embodiments, continue from step 504 of the method 500. In such embodiments, the step 704 may be performed when the imaging source 138 and/or the imaging detector 140 comprise an adjustable collimator, which may be the same as or similar to the collimator 144. The adjustable collimator may be a collimator whose orientation can be changed to adjust the quantity and/or direction in which radiation is emitted or detected. For example, one or more windows of the adjustable collimator may be slidable or movable to reduce or increase the quantity of radiation emitted or detected. Additionally or alternatively, the windows may form an aperture through which the radiation is emitted or detected, with such aperture being moveable based on the positioning of the windows.

The step 704 may include the processor 104 determining the orientation of the adjustable collimator. For example, the processor 104 may access the database 130 to retrieve information (e.g., a surgical plan) related to the orientation of the adjustable collimator. In other embodiments, the processor 104 may receive the adjustable collimator orientation information from a user (e.g., a physician, a surgical staff member, etc.). For instance, the user may enter information (e.g., via the user interface 110) about the current position of the adjustable collimator.

The processor 104 may adjust the virtual collimator 316 to match the current orientation of the adjustable collimator. The adjustment of the virtual collimator 316 may be such that, when the virtual collimator 316 is overlaid on an image, the virtual collimator 316 depicts the same collimation as the adjustable collimator would if the imaging source or imaging detector were used to capture the image. In some examples, the adjustable collimator is positioned in a first orientation, and the processor 104 may change the shape of the virtual collimator 316 to reflect the first orientation of the adjustable collimator. The processor 104 may use transformation 124 to transform the coordinates associated with the adjustable collimator into corresponding pixel locations of the virtual collimator 316. In some embodiments, the transformation 124 may be performed iteratively until the virtual collimator 316 is within a threshold distance of the first orientation (e.g., less than a 1% difference in relative position, less than 2% difference in relative position, etc.).

The method 700 also comprises rendering, to a display, a virtual representation of the virtual collimator (step 708). Once the virtual collimator 316 is adjusted to reflect the adjustable collimator, the virtual representation of the virtual collimator 316 may be rendered to the display, such as the user interface 110. In some embodiments, the virtual collimator 316 may be rendered during each iteration of the adjustment of the virtual collimator 316. In some embodiments, the visual representation of the virtual collimator 316 may include visual indicators to assist users with identifying the virtual collimator 316. Non-limiting examples of visual indicators include an outline of the border(s) of the virtual collimator 316, shading over the regions of the image blocked by the virtual collimator 316, combinations thereof, and the like.

The present disclosure encompasses embodiments of the method 700 that comprise more or fewer steps than those described above, and/or one or more steps that are different than the steps described above.

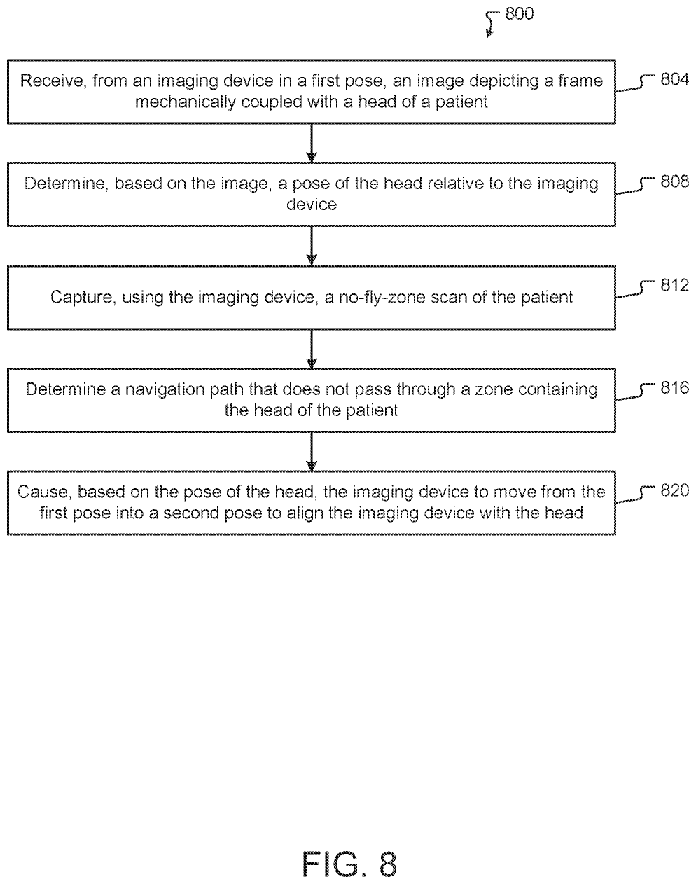

FIG. 8 depicts a method 800 that may be used, for example, to identify the pose of a patient's head and navigate components based on the pose of the patient's head.

The method 800 (and/or one or more steps thereof) may be carried out or otherwise performed, for example, by at least one processor. The at least one processor may be the same as or similar to the processor(s) 104 of the computing device 102 described above. The at least one processor may be part of a navigation system (such as a navigation system 118). A processor other than any processor described herein may also be used to execute the method 800. The at least one processor may perform the method 800 by executing elements stored in a memory such as the memory 106. The elements stored in memory and executed by the processor may cause the processor to execute one or more steps of a function as shown in method 800. One or more portions of a method 800 may be performed by the processor executing any of the contents of memory, such as an image processing 120, a segmentation 122, a transformation 124, and/or a registration 128.

The method 800 comprises receiving, from an imaging device in a first pose, an image depicting a frame mechanically coupled with a head of a patient (step 804). In some embodiments, the imaging device may be similar to or the same as the camera 136, the imaging source 138, and/or the imaging detector 140. The frame may be similar to or the same as the stereotactic frame 404. As discussed above, the initial image of the head of the patient may include a depiction of the stereotactic frame 404, which may be mounted, attached to, or otherwise mechanically coupled with the patient's head.

The method 800 also comprises determining, based on the image, a pose of the head relative to the imaging device (step 808). The pose of the head relative to the imaging device may be determined by the processor 104 using segmentation 122 to segment the captured image and identify navigation markers (e.g., navigation markers 408A-408C) attached to the frame. The processor 104 may then determine the position of the navigation markers relative to the imaging device that captured the image. Based on the known orientation of the navigation markers relative to the frame and the known orientation of the frame relative to the patient's head, the navigation system 118 may be able to determine the pose of the head relative to the imaging device. In some embodiments, the processor 104 may use registration 128 to register one or more coordinates of the frame (e.g., stereotactic frame 404) to a coordinate system associated with the imaging device. In some embodiments, the coordinate system may be a global coordinate system shared by one or more components of the platform 200 and that is used by the navigation system 118 when navigating tracked components.

The method 800 also comprises capturing, using the imaging device, a no-fly-zone scan of the patient (step 812). The no-fly-zone scan may be performed by the imaging device, and may include data associated with the patient. For example, the imaging device may perform a scan of the patient (e.g., from the patient's head to the patient's feet), and the processor 104 may use the data in determining zones that include the patient and zones that do not include the patient.

The method 800 also comprises determining a navigation path that does not pass through a zone containing the head of the patient (step 816). Continuing from the step 812, the processor 104 may further define zones including the patient as zones through which the navigation system 118 is prohibited from navigating components. For example, a first zone may include the patient's head, and the processor 104 may prevent the navigation system 118 from navigating tracked objects (e.g., surgical instruments or tools, imaging devices, etc.) through the first zone.

The method 800 also comprises causing, based on the pose of the head, the imaging device to move from the first pose into a second pose to align the imaging device with the head (step 820). Once the pose of the patient's head is known relative to the imaging device, the imaging device may be navigated by the navigation system 118 while avoiding collision with the patient. Since the navigation system 118 knows the position of both the imaging device and the patient in the same coordinate system, the navigation system 118 can avoid collisions between the two while moving the imaging device (or, more generally, while moving any tracked component relative to the patient's head). The navigation system 118 may navigate components through zones that have been labeled by the processor 104 as zones that do not contain portions of the patient, while avoiding navigation paths that cross into zones that have been identified as being or containing portions of the patient.

The present disclosure encompasses embodiments of the method 800 that comprise more or fewer steps than those described above, and/or one or more steps that are different than the steps described above.

As noted above, the present disclosure encompasses methods with fewer than all of the steps identified in FIGS. 5, 6, 7, and 8 (and the corresponding description of the methods 500, 600, 700, and 800), as well as methods that include additional steps beyond those identified in FIGS. 5, 6, 7, and 8 (and the corresponding description of the methods 500, 600, 700, and 800). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method, comprising:
receiving, from an imaging device in a pose relative to a patient, a first two-dimensional image depicting a first region of the patient from a perspective of the pose;
overlaying, over the first two-dimensional image, a virtual collimator to produce a second two-dimensional image, the second two-dimensional image depicting the first region as seen by a radiation source from the perspective of the pose; and
segmenting the second two-dimensional image into at least two segments, wherein a first segment of the at least two segments is subject to radiation produced by the radiation source while the radiation source is in the pose, and wherein a second segment of the at least two segments is subject to less radiation than the first segment of the at least two segments while the radiation source is in the pose.

2. The method of claim 1, further comprising:
causing the radiation source to move into the pose from a different position that is unaligned with the pose; and
causing the radiation source to emit the radiation after the radiation source has been moved into the pose.

3. The method of claim 1, further comprising:
rendering, to a display, a virtual representation of at least one of the first segment and the second segment.

4. The method of claim 3, wherein the virtual representation of the at least one of the first segment and the second segment comprises outlines of the at least one of the first segment and the second segment rendered over the second two-dimensional image.

5. The method of claim 1, further comprising:
transforming, based on the pose of the imaging device and a second pose of the radiation source, at least one of the first segment into a third segment that is subject to radiation produced by the radiation source while the radiation source is in the second pose and the second segment into a fourth segment that is subject to less radiation than the third segment while the radiation source is in the second pose.

6. The method of claim 5, further comprising:
causing the radiation source to move into the second pose; and
causing the radiation source to emit radiation after the radiation source has been moved into the second pose.

7. The method of claim 5, wherein the transforming comprises:
registering at least one of coordinates associated with a boundary of the first segment and coordinates associated with a boundary of the second segment into a coordinate system associated with the radiation source.

8. The method of claim 1, wherein the radiation source comprises an adjustable collimator.

9. The method of claim 8, further comprising:
adjusting, based on an orientation of the adjustable collimator, a shape of the virtual collimator.

10. The method of claim 9, further comprising:
rendering, to a display, a virtual representation of the virtual collimator.

11. A system, comprising:
a processor; and
a memory storing data thereon that, when processed by the processor, cause the processor to:
receive, from an imaging device in a first pose, an image depicting a frame mechanically coupled with a head of a patient;
determine, based on the image, a pose of the head relative to the imaging device; and
cause, based on the pose of the head, the imaging device to move from the first pose into a second pose to align the imaging device with the head.

12. The system of claim 11, wherein the frame comprises two or more navigation markers disposed on the frame.

13. The system of claim 12, wherein the data further cause the processor to:
determine, based on the two or more navigation markers, a pose of the frame.

14. The system of claim 13, wherein the two or more navigation markers comprise optical navigation markers capable of being identified in the image.

15. The system of claim 13, wherein the data further cause the processor to:
register one or more coordinates associated with the frame to a coordinate system associated with the imaging device.

16. The system of claim 11, wherein the data further cause the processor to:
capture, using the imaging device, a no-fly-zone scan of the patient.

17. The system of claim 16, wherein the no-fly-zone scan comprises a first zone through which the imaging device is moveable and a second zone through which the imaging device avoids moving.

18. The system of claim 17, wherein causing the imaging device to move from the first pose to the second pose comprises:
determining a navigation path that does not pass through the second zone.

19. The system of claim 17, wherein the second zone comprises at least one of the head of the patient and the frame.

20. The system of claim 11, wherein the imaging device comprises an O-arm.

21. A system, comprising:
a processor; and
a memory storing data thereon that, when processed by the processor, cause the processor to:
receive, from an imaging device in a first pose relative to a patient, a first two-dimensional image depicting a first region of the patient from a perspective of the first pose;
overlay, over the first two-dimensional image, a virtual collimator to produce a second two-dimensional image, the second two-dimensional image depicting a first region of the patient as seen by a radiation source from the perspective of the first pose;
segment, the second two-dimensional image into at least two segments, wherein a first segment of the at least two segments is subject to radiation produced by the radiation source while the radiation source is in the first pose, and wherein a second segment of the at least two segments is subject to less radiation than the first segment of the at least two segments while the radiation source is in the first pose;
determine, based on the first two-dimensional image, a pose of a head of the patient relative to the imaging device; and
cause, based on the pose of the head, the imaging device to move from the first pose to a second pose to align the imaging device with the head.

22. A system, comprising:
a first imaging device;
a second imaging device;
a processor; and
a memory coupled to the processor and storing data thereon that, when processed by the processor, enable the processor to:
receive, from the first imaging device in a pose relative to a patient, a first two-dimensional image depicting a first region of the patient from a perspective of the pose;
overlay, over the first two-dimensional image, a virtual collimator to produce a second two-dimensional image, the second two-dimensional image depicting the first region as seen by a radiation source from the perspective of the pose; and
segment the second two-dimensional image into at least two segments, wherein a first segment of the at least two segments is subject to radiation produced by the radiation source while the radiation source is in the pose, and wherein a second segment of the at least two segments is subject to less radiation than the first segment of the at least two segments while the radiation source is in the pose.

23. The system of claim 22, wherein the data further enable the processor to:
capture, using the first imaging device and the second imaging device, a three-dimensional image depicting the patient; and
segment the three-dimensional image into a plurality of segments, wherein a first segment of the plurality of segments contains the first region of the patient.

24. The system of claim 23, wherein the data further enable the processor to:
determine, based on the first segment of the plurality of segments, a location of the first region of the patient relative to the first imaging device; and
cause, based on the location of the first region of the patient, the first imaging device to move into the pose relative to the patient.

25. The system of claim 24, wherein the data further enable the processor to:
cause the radiation source to move into the pose from a different position that is unaligned with the pose; and
cause the radiation source to emit the radiation after the radiation source has been moved into the pose.

26. The system of claim 24, wherein the data further enable the processor to:
render, to a display, a virtual representation of at least one of the first segment and the second segment of the at least two segments.

27. The system of claim 26, wherein the virtual representation of the at least one of the first segment and the second segment of the at least two segments comprises outlines of the at least one of the first segment and the second segment of the at least two segments rendered over the second two-dimensional image.

28. The system of claim 24, wherein the data further enable the processor to:
transform, based on the pose of the first imaging device and a second pose of the radiation source, at least one of the first segment of the at least two segments into a third segment that is subject to radiation produced by the radiation source while the radiation source is in the second pose and the second segment of the at least two segments into a fourth segment that is subject to less radiation than the third segment while the radiation source is in the second pose.

29. The system of claim 28, further comprising:
causing the radiation source to move into the second pose; and
causing the radiation source to emit radiation after the radiation source has been moved into the second pose.

30. The system of claim 28, wherein the transforming comprises:
registering at least one of coordinates associated with a boundary of the first segment of the at least two segments and coordinates associated with a boundary of the second segment of the at least two segments into a coordinate system associated with the radiation source.

31. The system of claim 24, wherein the radiation source comprises an adjustable collimator.

32. The system of claim 31, wherein the data further enable the processor to:
adjust, based on an orientation of the adjustable collimator, a shape of the virtual collimator.

33. The system of claim 32, wherein the data further enable the processor to:
render, to a display, a virtual representation of the virtual collimator.
